(19) United States
(12) Patent Application Publication
(10) Pub. No.: US 2020/0002660 A1
(43) Pub. Date: Jan. 2, 2020

(54) Portable Device and Methods for Efficient Production of Microbes

(71) Applicant: LOCUS IP COMPANY, LLC, Solon, OH (US)

(72) Inventors: Sean FARMER, North Miami Beach, FL (US); Ken ALIBEK, Solon, OH (US); Tyler DIXON, Kent, OH (US)

(21) Appl. No.: 16/484,488

(22) PCT Filed: Feb. 12, 2018
(86) PCT No.: PCT/US2018/017814

§ 371 Date: Aug. 8, 2019

Related U.S. Application Data
(60) Provisional application No. 62/457,445, filed on Feb. 10, 2017.

(51) Int. Cl. C12M 1/00; C12M 3/04; C12M 3/06

(52) U.S. Cl. CPC C12M 23/52; C12M 23/16; C12M 27/20; C12M 27/10

(57) ABSTRACT

Provided are devices and methods for producing microbe-based compositions that can be used in the oil and gas industry, environmental cleanup, as well as for other applications. The devices and methods can produce scalable, submerged yeast cultures for inoculating larger-scale, on-site fermentation systems. A device can include a rotatable drum mounted on a support frame and a motor connected to the drum and causing the drum to rotate.

### CROSS-REFERENCE TO RELATED APPLICATION

This application claims the benefit of U.S. Provisional Application Ser. No. 62/457,445, filed Feb. 10, 2017, which is incorporated herein by reference in its entirety, including any figures, tables, and drawings.

### FIELD OF THE INVENTION

The present invention relates to devices and methods for producing microbe-based compositions that can be used in, for example, the oil industry, agriculture, aquaculture, mining, waste treatment and bioremediation.

### BACKGROUND OF THE INVENTION

Cultivation of microorganisms such as bacteria, yeast and fungi is important for the production of a wide variety of useful bio-preparations. Microorganisms play crucial roles in, for example, food industries, pharmaceuticals, agriculture, mining, environmental remediation, and waste management.

An enormous potential exists for the use of fungi in a broad range of industries. The restricting factor in commercialization of fungi-based products has been the cost per propagule density, where it is particularly expensive and unfeasible to apply fungal products to large scale operations at sufficient concentrations to see the benefits.

Two principle forms of cultivation of microorganisms exist: submerged cultivation and surface cultivation. Bacteria, yeasts and fungi can all be grown using either method. Both cultivation methods require a nutrient medium for the growth of the microorganisms. The nutrient medium, which can either be in a liquid or a solid form, typically includes a carbon source, a nitrogen source, salts and appropriate additional nutrients and microelements. The pH and oxygen levels are maintained at values suitable for a given microorganism.

Agriculture and the oil industry are two industries where microbes could play highly beneficial roles if they could be made more readily available and, preferably, in a more active form.

As crude oil flows through a well, substances in the crude oil often collect on the surfaces of the production lines, causing a reduction in flow and even stopping production all together. A variety of different chemicals and equipment are utilized to inhibit or prevent and remediate this issue, but there is a need for better products and methods, especially more environmentally friendly methods that have improved effectiveness and reduced toxicity.

In order to boost yields and protect crops against pathogens, pests, and disease, farmers have relied heavily on the use of synthetic chemicals and chemical fertilizers; however, when overused or improperly applied, these substances can run off into surface water, leach into groundwater, and evaporate into the air. Even when properly used, the over-dependence and long-term use of certain chemical fertilizers and pesticides deleteriously alter soil ecosystem, reduce stress tolerance, increase pest resistance, and impede plant and animal growth and vitality.

While wholesale elimination of chemicals is not feasible at this time, farmers are increasingly embracing the use of biological measures as viable components of Integrated Nutrient Management and Integrated Pest Management programs. For example, in recent years, biological control of nematodes has created great interest. This method utilizes biological agents such as live microbes, bio-products derived from these microbes, and combinations thereof as pesticides. These biological pesticides have important advantages over other conventional pesticides. For example, they are less harmful compared to the conventional chemical pesticides. They are more efficient and specific. They often biodegrade quickly, leading to less environmental pollution.

The use of biopesticides and other biological agents has been greatly limited by difficulties in production, transportation, administration, pricing and efficacy. For example, many microbes are difficult to grow and subsequently deploy to agricultural and oil production systems in sufficient quantities to be useful. This problem is exacerbated by losses in viability and/or activity due to processing; formulating; storage; stabilizing prior to distribution; sporulation of vegetative cells as a means of stabilizing; transportation, and application.

Microbe-based compositions could help meet these needs if more efficient cultivation methods for mass production of microorganisms and microbial metabolites were available.

### SUMMARY OF THE INVENTION

The present invention provides devices and methods for producing microbe-based compositions that can be used in the oil and gas industry, agriculture, bioremediation, aquaculture, and many other applications. Specifically, the subject invention provides methods and materials for efficient cultivation of microorganisms and production of microbial growth by-products. The subject invention also provides devices for such cultivation and production.

More specifically, the present invention provides a fermentation device that can be used and transported at low cost without requiring special training or skill. In specific embodiments, the device and methods are used to cultivate yeast and fungi inocula, which can then be used in larger fermentation systems. In certain embodiments, the device and methods are used for the production of Starmerella bombicola yeast inocula.

In one embodiment, the device of the subject invention comprises a rotating drum supported by a frame, which can have wheels. Rotation of the drum is achieved using a motor (e.g., an electric motor) connected to a power supply (e.g., the device can have a battery or a power cord for connecting to an external power supply). The drum can also be connected to an aeration system, for example an aeration system comprising an air pump. This serves to provide air to the surface of the culture inside the drum and can also serve as a means of regulating the internal temperature.

Baffles can be attached to the inner surface of the drum, to aid in the agitation and aeration of the culture. While the drum is rotating, the culture is mixed therein and oxygenated by ambient air as well as air supplied by the aeration system.

In preferred embodiments, the device operates continuously throughout the process of cultivation. The device can be operated for as long as necessary to produce a sufficient volume of culture, depending on the particular species of microorganism being produced. For example, the mixing device can be run continuously for 1, 2, 3, 4, 5 or more days (or any portion thereof).

Advantageously, the device can be effectively self-sterilizing. For example, microorganisms cultivated within the mixing device can be strains that produce antimicrobial metabolites or byproducts, such as biosurfactants. Thus, the microbe culture itself can provide control of unwanted microorganisms inside the drum, simultaneously with cultivation of the desired microorganisms.

In preferred embodiments, the subject invention provides cultivation methods that simplify production and facilitate portability of useful microbe-based compositions and products. The methods provide for submerged cultivation of microbe compositions suitable for inoculating large-scale fermentation systems.

The inoculum produced by the subject device and method can be used to inoculate a fermentation system present on-site for production of large quantities of microbe-based compositions. In preferred embodiments, the subject device and methods can also be used on-site, in such a way that the inoculum culture can be transferred directly from the device to the on-site fermentation system.

Advantageously, the subject invention reduces the capital and labor costs of producing microorganisms and their metabolites. Furthermore, the cultivation process of the subject invention reduces or eliminates the need to concentrate or otherwise process microbes after completing cultivation.

Portability can result in significant cost savings as inoculums for microbe-based compositions can be produced at, or near, the site of intended inoculation. Advantageously, inoculum can be produced on-site using locally-sourced materials if desired, thereby reducing the logistical obstacles and costs of transporting and shipping. Furthermore, the end products produced by scaling the inoculum can include viable microbes at the time of application.

Compositions produced by the present invention can be used to inoculate large-scale fermentation systems for use in a wide variety of petroleum industry applications. These applications include, but are not limited to, enhancement of crude oil recovery; reduction of oil viscosity; paraffin removal from rods, tubing, liners, and pumps; petroleum equipment corrosion inhibition or prevention; fracturing fluids; reduction of H₂S concentration in extracted crude oil; as well as tank, flowline and pipeline cleaning.

### BRIEF DESCRIPTION OF THE FIGURE

FIG. 1 shows a device according to an embodiment of the present invention.

### DETAILED DESCRIPTION OF THE INVENTION

The present invention provides devices and methods for producing microbe-based compositions that can be used in the oil and gas industry, agriculture, bioremediation, aquaculture, and many other applications. Specifically, the subject invention provides methods and materials for efficient cultivation of microorganisms and production of microbial growth by-products. The subject invention also provides devices for such cultivation and production.

More specifically, the present invention provides a mobile fermentation device that can be used and transported at low cost without requiring special training or skill.

In specific embodiments, the device and methods are used to produce yeast and fungi inocula. In certain embodiments, the device and methods are used for the production of Starmerella bombicola yeast inocula.

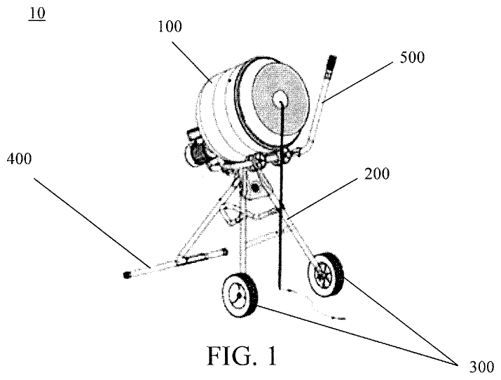

In one embodiment, the device of the subject invention comprises a rotating drum supported by a frame. The frame can have wheels for ease of movement, though this is not necessary. For example, the device can include two wheels on one side with no wheels on the other side so that the device can be tipped for transport using the wheels and set down to remain in place (as depicted in FIG. 1). Alternatively, the device can include three or more wheels. Rotation of the drum is achieved using a motor. The motor can be powered by, for example, electricity or gas. Preferably, the motor is an electric motor that can be connected to a power supply. For example, the device can include a battery as a power supply or the device can derive power from an external source (e.g., via a power cord or through wireless power transfer).

The drum can also be connected to an aeration system comprising, for example, an air pump. This serves to provide air to the surface of the culture inside the drum and can also serve as a means of regulating the internal temperature.

Baffles can be attached to the inner surface of the drum, to aid in the agitation and aeration of the culture. While the drum is rotating, the culture is mixed therein and oxygenated by ambient air as well as air supplied by the aeration system.

In preferred embodiments, the device operates continuously throughout the process of cultivation. The device can be operated for as long as necessary to produce a sufficient volume of culture, depending on the particular species of microorganism being produced. For example, the mixing device can be run continuously for 1, 2, 3, 4, 5 or more days (or any portion thereof).

The device of the present invention can be scaled depending on the intended use. For example, the drum can range in volume from a few liters to several hundred liters or more.

Advantageously, the device can be effectively self-sterilizing. For example, microorganisms cultivated within the mixing device can be strains that produce antimicrobial metabolites or byproducts, such as biosurfactants. Thus, the microbe culture itself can provide control of unwanted microorganisms inside the drum, simultaneously with cultivation of the desired microorganisms. Alternatively, or additionally, the device can be sterilized with external means, for example, a sterilizing agent such as hydrogen peroxide.

In preferred embodiments, the subject invention provides cultivation methods that simplify production and facilitate portability of useful microbe-based compositions and products. The methods provide for submerged cultivation of microbe compositions suitable for inoculating large-scale fermentation systems.

In certain embodiments, the method comprises adding to the drum of the fermentation device at least one type of microorganism, and, optionally, nutrients for the microorganisms; and allowing the mixing device to operate until a sufficient amount of inoculum has been produced. The nutrients can include, for example, one or more carbon sources, proteins, fats, nitrogen sources, trace elements, and/or growth factors (e.g., vitamins, pH regulators).

The inoculum produced by the subject method can be used to inoculate a fermentation system present on-site for production of large quantities of microbe-based compositions. In preferred embodiments, the subject device and methods can be used on-site in such a way that the inoculum culture can be transferred directly from the device to a larger-scale on-site fermentation system.

In one embodiment, the subject invention further provides an inoculum composition comprising at least one type of microorganism and/or at least one microbial metabolite produced by the microorganism that has been grown using the device of the subject invention. The microorganisms in the composition may be in an active or inactive form. The composition may also be in a dried form or a liquid faint.

Advantageously, the subject invention reduces the capital and labor costs of producing microorganisms and their metabolites. Furthermore, the cultivation process of the subject invention reduces or eliminates the need to concentrate or otherwise process microbes after completing cultivation.

Portability can result in significant cost savings as inoculums for microbe-based compositions can be produced at, or near, the site of intended inoculation. Advantageously, inoculum can be produced on-site using locally-sourced materials if desired, thereby reducing the logistical obstacles and costs of transporting and shipping. Furthermore, the end products produced by scaling the inoculum can include viable microbes at the time of application, which can increase product effectiveness.

Thus, in certain embodiments, the subject invention harnesses the power of naturally-occurring local microorganisms and their metabolic by-products. Use of local microbial populations can be advantageous in settings including, but not limited to, environmental remediation (such as in the case of an oil spill), animal husbandry, aquaculture, forestry, pasture management, turf management, horticultural ornamental production, waste disposal and treatment, mining, oil recovery, and human health, including in remote locations.

Compositions produced by the present invention can be used to inoculate large-scale fermentation systems for use in a wide variety of petroleum industry applications. These applications include, but are not limited to, enhancement of crude oil recovery; reduction of oil viscosity; paraffin removal from rods, tubing, liners, and pumps; petroleum equipment corrosion inhibition or prevention; fracturing fluids; reduction of H₂S concentration in extracted crude oil; as well as tank, flowline and pipeline cleaning.

### Selected Definitions

As used herein, “microbe-based composition” means a composition that comprises components that were produced as the result of the growth of microorganisms or other cell cultures. Thus, the microbe-based composition may comprise the microbes themselves and/or by-products of microbial growth. The cells may be in a vegetative state or in spore form, or a mixture of both. The cells may be planktonic or in a biofilm form, or a mixture of both. The by-products of growth may be, for example, metabolites, cell membrane components, expressed proteins, and/or other cellular components. The cells may be intact or lysed. In preferred embodiments, the cells are in the vegetative state and are present, with broth in which they were grown, in the microbe-based composition. The cells may be present at, for example, a concentration of 1×10⁴, 1×10⁵, 1×10⁶, 1×10⁷, 1×10⁸, 1×10⁹, 1×10¹⁰, or 1×10¹¹ or more cells per milliliter of the composition

The subject invention further provides “microbe-based products,” or “cultivation products,” which are products that are to be applied in practice to achieve a desired result. The microbe-based product can be simply the microbe-based composition harvested from the microbe cultivation process. Alternatively, the microbe-based product may comprise further ingredients that have been added. These additional ingredients can include, for example, buffers, appropriate carriers, such as water, added nutrients to support further microbial growth, and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

The term “inoculum” is encompassed within the term “microbe-based product.” As used herein, inoculum means a microbe-based product that can be used, for example, as a seed culture to inoculate a larger scale fermentation system or process. The inoculum can be scaled in such a fermentation system to produce desired quantities of microbe-based compositions and products.

As used herein, “on-site fermentation system” refers to a system used for producing microbe-based compositions and/or products at or near to the site of application of these microbe-based compositions and/or products.

As used herein, “harvested” refers to removing some or all of the microbe-based composition from a growth vessel.

As used herein, the term “control” used in reference to the activity produced by the biosurfactants (on other active agent) or biosurfactant-producing microorganisms extends to the act of killing, disabling or immobilizing pests or otherwise rendering the pests substantially incapable of causing harm.

### Mixing Device Design and Operation

FIG. 1 depicts a fermentation device according to an embodiment of the present invention. Referring to FIG. 1, the device 10 can include a rotating drum 100 supported by a frame 200. The frame 200 can have wheels 300 for ease of movement, though this is not necessary. For example, the device can include two wheels 300 on one side with no wheels on the other side 400 so that the device can be tipped for transport using the wheels and set down to remain in place. Alternatively, the device can include three or more wheels 300 (either such that all points of contact with the ground are wheels, or while still including a section 400 with no wheels). The wheels 300 can have wheel locks to hold the device in place when not in transport, particularly in the case where all points of contact with the ground are wheels. Rotation of the drum 100 is achieved using a motor. The motor can be powered by, for example, electricity or gas. Preferably, the motor is an electric motor that can be connected to a power supply. For example, the device can include a battery as a power supply or the device can derive power from an external source (e.g., via a power cord or through wireless power transfer). The device 10 may be equipped with a means for adjusting the angle of the drum 100. Such a means can include, for example, a lever 500 and/or a hinge or other rotatable support on the frame 200.

In one embodiment, the drum 100 of the device 10 is a closable rotating drum for holding, mixing, and growing a submerged culture inoculum. The drum may be made from, for example, glass, one or polymers, one or more metals, one or more metal alloys, and/or combinations thereof.

The drum 100 can be mounted on a support frame 200. The support frame 200 can have wheels 300, facilitating easy transport of the entire device without requiring extensive skill, training, cost, or time. The wheels 300 can be made of, for example, one or more polymers, rubber, or any durable material suitable for movement across a variety of landscapes, such as those found in agricultural and oil extraction environments. The frame 200 can be made of, for example, glass, one or polymers, one or more metals, one or more metal alloys, and/or combinations thereof.

The drum is operably engaged with a motor, such as an electric motor, which is connected to a power supply. The motor enables the drum to rotate continuously at a speed of, for example, 10 to 30 rpm and, more preferably, 15-25 rpm.

The drum can also be connected to an aeration system comprising, for example, an air pump. The air pump provides air to the inside of the drum, thereby aerating the surface of the moving culture inside the drum. While the drum is rotating, the culture is mixed therein and oxygenated by the air supplied by the aeration system. In some embodiments, the air can be heated or cooled to help regulate the internal temperature of the drum and culture environment.

The angle of the axis of the drum with respect to the ground can be from 0° to 90°. The angle is preferably less than 90° in order to increase the surface area of the culture broth within the drum. The angle may be horizontal (i.e., 0°), or close to horizontal. The angle may be, for example, from about 5° to about 75°, or from about 10° to about 60°. The device may be equipped with a means for adjusting the angle.

Along with optimizing the angle of the axis of the drum, the shape of the drum is also preferably optimized such that a maximum surface area of culture is exposed to the air supply during the cultivation process. The drum can be shaped like, for example, a cylinder, or any type of modified cylinder, though embodiments are not limited thereto. Modified cylinders can include tapered cylinders, can-shaped cylinders, or cylinders having a wider diameter at the middle than at either end.

Additionally, baffles can be present on the inner surface of the drum to aid in the proper agitation and aeration of the culture. Preferably, 3 to 4 baffles are evenly, or roughly evenly, spaced around the inner circumference of the drum and aligned so they are parallel to the axis of the drum's rotation. Alternatively, the baffles can be disposed such that they are perpendicular to the axis of the drum's rotation.

In preferred embodiments, the device operates continuously throughout the process of cultivation. The device can be operated for as long as necessary to produce a sufficient volume of culture, depending on the particular microbe species being produced. For example, the mixing device can be run continuously for multiple days. In specific embodiments, the mixing device is run continuously for 1, 2, 3, 4, or up to 5 days or more, or any portion thereof.

In one embodiment, the mixing device is a mobile or portable bioreactor that may be provided for on-site production of an inoculum including a suitable amount of a desired strain of microorganism. The amount of liquid culture inoculum produced can be, for example, 2 to 500 liters, 5 to 250 liters, 10 to 100 liters, 15 to 75 liters, 20 to 50 liters, or 35 to 40 liters. Because the inoculum is generated on-site of the application, without resort to stabilization, preservation, storage and transportation processes of conventional production, a much higher density of live microorganisms may be generated, thereby requiring a much smaller volume of the microorganism composition for use in an on-site fermentation system. This allows for a scaled-down bioreactor (e.g., smaller fermentation tanks, smaller supplies of starter material, nutrients, pH control agents, and de-foaming agent, etc.) that facilitates the mobility and portability of the system.

The device of the present invention can be scaled depending on the intended use. For example, the drum can range in volume from a few liters to several hundred liters, depending on how much inoculum will be needed to inoculate a specific fermentation system. The drum may be, for example, from 1 liter to 5,000 liters or more. Typically, the drum can be from 10 to 1,500 liters, preferably from 50 to 500 liters, and more preferably from 100 to 200 liters.

In one embodiment, the device has functional controls/sensors or may be connected to functional controls/sensors to measure important factors in the cultivation process, such as pH, oxygen, pressure, temperature, agitator shaft power, humidity, viscosity, and/or microbial density and/or metabolite concentration.

In one embodiment, the device has its own controls and measuring systems for at least temperature and pH. In addition to monitoring and controlling temperature and pH, the drum may also have the capability for monitoring and controlling, for example, dissolved oxygen, agitation, foaming, purity of microbial cultures, production of desired metabolites and the like.

In a further embodiment, the device may also be able to monitor the growth of microorganisms inside the vessel (e.g., measurement of cell number and growth phases). Alternatively, a daily sample may be taken from the vessel and subjected to enumeration by techniques known in the art, such as dilution plating technique. Dilution plating is a simple technique used to estimate the number of bacteria in a sample. The technique can also provide an index by which different environments or treatments can be compared.

In one embodiment, cultivation medium, air, and equipment used in the method and cultivation process are sterilized. The cultivation equipment such as the reactor/vessel may be separated from, but connected to, a sterilizing unit, e.g., an autoclave. The cultivation equipment may also have a sterilizing unit that sterilizes in situ before starting the inoculation, e.g., by using steam. The air can be sterilized by methods know in the art. For example, the ambient air can pass through at least one filter before being supplemented into the vessel. In other embodiments, the medium may be pasteurized or optionally no heat at all added, where the use of low water activity and low pH may be exploited to control bacterial growth.

Before cultivation the drum can be washed with a sterilizing agent, such as a hydrogen peroxide solution (e.g., from 1.0% to 3.0% hydrogen peroxide); this can be done before or after a hot water rinse at, e.g., 80-90 degrees Celsius to inhibit or prevent contamination. The culture medium components (e.g., the carbon source, water, lipid source, micronutrients, etc.) can also be temperature decontaminated and/or hydrogen peroxide decontaminated (potentially followed by neutralizing the hydrogen peroxide using an acid such as HCl, H₂SO₄, etc.).

Advantageously, the device can also be self-sterilizing. For example, microorganisms chosen for cultivation within the mixing device can be strains known to produce antimicrobial metabolites or byproducts, such as biosurfactants. Thus, the microbe culture itself can provide control of unwanted microorganisms inside the drum, simultaneously with cultivation of the desired microorganisms.

The culturing temperature utilized according to the present invention can be, for example, from about 25 to 40 degrees Celsius, although the process may operate outside of this range. The microbe can be cultured in a pH range from about 2 to 10 and, more specifically, at a pH range of from about 3 to 5 (by manually or automatically adjusting pH using bases, acids, and buffers; e.g., HCl, KOH, NaOH, H₃PO₄). The invention can also be practiced outside of this pH range.

Yeast cultivation can start at a first pH (e.g., a pH of 4.0 to 4.5) and later change to a second pH (e.g., a pH of 3.2-3.5) for the remainder of the process to help avoid contamination as well as to produce other desirable results (the first pH can be either higher or lower than the second pH). Preferable results may be achieved by keeping the dissolved oxygen concentration above 10, 15, 20, or 25% of saturation during cultivation. In one embodiment, the inoculum does not need to be further processed after cultivation (e.g., yeast, metabolites, and remaining carbon sources do not need to be separated from the sophorolipids). The physical properties (e.g., viscosity, density, etc.) can also be adjusted using various chemicals and materials that are known in the art.

One or more antimicrobial substances can be added to the culture medium (e.g., streptomycin, oxytetracycline, sophorolipid, and rhamnolipid) to further inhibit or prevent contamination, before, during, or after fermentation. One or more organic and inorganic nitrogen sources can be added to the medium (e.g., protein, amino acids, yeast extracts, yeast autolysates, ammonia or ammonium salts, urea, corn peptone, casein hydrolysate, and soybean protein).

### Microorganisms

The microorganisms grown according to the subject invention can be, for example, bacteria, yeast, fungi or multicellular organisms. In preferred embodiments, the microorganism is a yeast. In particularly preferred embodiments, the microbes are of the Starmerella clade strains.

In one embodiment, the microorganisms are bacteria, including gram-positive and gram-negative bacteria. These bacteria may be, but are not limited to, for example, Escherichia coli, Rhizobium (e.g., Rhizobium japonicum, Sinorhizobium meliloti, Sinorhizobium fredii, Rhizobium leguminosarum biovar trifolii, and Rhizobium etli), Bradyrhizobium (e.g., Bradyrhizobium japanicum, and B. parasponia), Bacillus (e.g., Bacillus subtilis, Bacillus firmus, Bacillus laterosporus, Bacillus megaterium, Bacillus amyloliquifaciens), Azobacter (e.g., Azobacter vinelandii, and Azobacter chroococcum), Arhrobacter (e.g. Agrobacterium radiobacter), Pseudomonas (e.g., Pseudomonas chlororaphis subsp. aureofaciens (Kluyver)), Azospirillium (e.g., Azospirillum brasiliensis), Azomonas, Derxia, Beijerinckia, Nocardia, Klebsiella, Clavibacter (e.g., C. xyli subsp. xyli and C. xyli subsp. cynodontis), cyanobacteria, Pantoea (e.g., Pantoea agglomerans), Sphingomonas (e.g., Sphingomonas paucimobilis), Streptomyces (e.g., Streptomyces griseochromogenes, Streptomyces qriseus, Streptomyces cacaoi, Streptomyces aureus, and Streptomyces kasugaenis), Streptoverticillium (e.g., Streptoverticillium rimofaciens), Ralslonia (e.g., Ralslonia eulropha), Rhodospirillum (e.g., Rhodospirillum rubrum), Xanthomonas (e.g., Xanthomonas campestris), Erwinia (e.g., Erwinia carotovora), Clostridium (e.g., Clostridium bravidaciens, and Clostridium malacusomae) and combinations thereof.

In one embodiment, the microorganism is a fungus (including yeast), including, but not limited to, for example, Starmerella, Mycorrhiza (e.g., vesicular-arbuscular mycorrhizae (YAM), arbuscular mycorrhizae (AM)), Mortierella, Phycomyces, Blakeslea, Thraustochytrium, Penicillium, Phythium, Entomophthora, Aureobasidium pullulans, Fusarium venenalum, Aspergillus, Trichoderma (e.g., Trichoderma reesei, T. harzianum, T. viride and T. hamatum), Rhizopus spp, endophytic fungi (e.g., Piriformis indica), Saccharomyces (e.g., Saccharomyces cerevisiae, Saccharomyces boulardii sequela and Saccharomyces torula), Debaromyces, Issalchenkia, Kluyveromyces (e.g., Kluyveromyces lactis, Kluyveromyces fragilis), Pichia spp (e.g., Pichia pastoris), and combinations thereof.

In one embodiment, a single type of microbe is grown in the mixing device. In alternative embodiments, multiple microbes, which can be grown together without deleterious effects on growth or the resulting product, can be grown together in the mixing device. There may be, for example, 2 to 3 or more different microbes grown in the device at the same time.

### Cultivation and Growth Medium

The subject invention provides methods for the efficient production of scalable submerged microbe cultures. The method can include providing all of the materials necessary for submerged cultivation process, although it is expected that freshwater would be supplied from a local source.

In one embodiment, the method comprises providing a viable yeast, or other microbe, inside the drum of the mixing device. A variety of strains can be included that are capable of accumulating significant amounts of glycolipid-biosurfactants. More specifically, the method can comprise adding one or more viable fungal strains capable of controlling pests, bioremediation, enhancing oil recovery and other useful purposes, e.g., Starmerella {Candida) bombicola, Candida apicola, Candida batistae, Candida floricola, Candida riodocensis, Candida stellate, Candida kuoi, Candida sp. NRRL Y-27208, Rhodotorula bogoriensis sp., Wickerhamiella domericqiae, as well as any other sophorolipid-producing strains of the Starmerella clade.

In one embodiment, the culture medium used according to the subject invention, may contain supplemental nutrients for the microorganism. Typically, these include carbon sources, proteins and/or fats, nitrogen sources, trace elements, and/or growth factors (e.g., vitamins, pH regulators). It will be apparent to one of skill in the art that nutrient concentration, moisture content, pH, and the like may be modulated to optimize growth for a particular microbe.

Each of the carbon source, lipid source, nitrogen source, and/or micronutrient source can be provided in an individual package that can be added to the drum of the mixing device at appropriate times during the cultivation process. Each of the packages can include several sub-packages that can be added at specific points (e.g., when yeast, pH, and/or nutrient levels go above or below a specific concentration) or times (e.g., after 10 hours, 20 hours, 30 hours, 40 hours, etc.) during the cultivation process.

The lipid source can include, for example, oils or fats of plant or animal origin which contain free fatty acids or their salts or their esters, including triglycerides. Examples of fatty acids include, but are not limited to, free and esterified fatty acids containing from 16 to 18 carbon atoms, hydrophobic carbon sources, palm oil, animal fats, coconut oil, oleic acid, soybean oil, sunflower oil, canola oil, stearic and palmitic acid. Other carbon sources can include one or more sugars such as glucose, xylose, mannose, sucrose, galactose, mannitol, sorbose, ribose, arbutin, raffinose, glycerol, erythritol, xylitol, gluconate, citrate, molasses, hydrolyzed starch, corn syrup, and hydrolyzed cellulosic material including glucose.

The method can comprise adding one or more micronutrient sources, such as potassium, magnesium, calcium, zinc and manganese, preferably as salts; phosphorous, such as from phosphates; and other growth stimulating components. One or more organic and inorganic nitrogen sources can be included such as proteins, amino acids, yeast extracts, yeast autolysates, ammonia or ammonium salts, urea, corn peptone, casein hydrolysate, and soybean protein.

The method can comprise adding one or more antimicrobial substances to inhibit or prevent contamination during cultivation (e.g., streptomycin, oxytetracycline, sophorolipid, and rhamnolipid). Furthermore, the method can include pre-cultivation decontamination materials such as bleach and hydrogen peroxide. The bleach and hydrogen peroxide can come in concentrated form and later be diluted at the fermentation site before use. For example, the hydrogen peroxide can be provided in concentrated form and be diluted to formulate 1.0% to 3.0% hydrogen peroxide (by weight or volume) for pre-rinse decontamination.

The method can also comprise adding one or more pH adjusting substances such as bases, acids, and buffers (e.g., HCl, KOH, NaOH, and/or H₃PO₄, H₂SO₄, etc). The pH adjustment can be accomplished by automatic means or it can be done manually. The automatic pH adjustment can include a pH probe and an electronic device to dispense the pH adjustment substances appropriately, depending on the pH measurements. The pH can be set to a specific number by a user or can be pre-programmed to change the pH accordingly throughout the cultivation process. If the pH adjustment is to be done manually, pH measurement tools known in the art can be used for manual testing.

A temperature sensor, such as a thermometer or thermocouple, can be used to monitor temperature, and the thermometer can be manual or automatic. An automatic thermometer can manage the heat and cooling sources appropriately to control the temperature throughout the cultivation process.

In one embodiment, the method includes supplementing the cultivation with a nitrogen source. The nitrogen source can be, for example, in an inorganic form such as potassium nitrate, ammonium nitrate ammonium sulfate, ammonium phosphate, ammonia, urea, and ammonium chloride, or an organic form such as proteins, and amino acids. These nitrogen sources may be used independently or in a combination of two or more.

The method can further comprise supplementing the cultivation with a carbon source. The carbon source is typically a carbohydrate, such as glucose, sucrose, lactose, fructose, trehalose, mannose, mannitol, and maltose; organic acids such as acetic acid, fumaric acid, citric acid, propionic acid, malic acid, malonic acid, and pyruvic acid; alcohols such as ethanol, propanol, butanol, pentanol, hexanol, isobutanol, and glycerol; fats and oils such as soybean oil, rice bran oil, olive oil, corn oil, sesame oil, and linseed oil; etc. These carbon sources may be used independently or in a combination of two or more.

In one embodiment, growth factors and trace nutrients for microorganisms are included in the medium. Inorganic nutrients, including trace elements such as iron, zinc, copper, manganese, molybdenum and cobalt may also be included in the medium.

In one embodiment, inorganic salts may also be included. Inorganic salts can be, for example, potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, calcium carbonate, sodium carbonate. These inorganic salts may be used independently or in a combination of two or more.

Advantageously, the method provides easy oxygenation of the growing culture with, for example, slow motion of air to remove low-oxygen containing air and introduction of oxygenated air. The oxygenated air may be ambient air supplemented periodically, such as daily.

In some embodiments, the method for cultivation may further comprise adding additional acids and/or antimicrobials in the liquid medium before, and/or during the cultivation process. Antimicrobial agents or antibiotics are used for inhibiting or preventing the culture from contamination. Additionally, antifoaming agents may also be added to inhibit or prevent the formation and/or accumulation of foam when gas is produced during cultivation and fermentation.

In one embodiment, the method for cultivation of microorganisms is carried out at about 5° to about 100° C., preferably, 15° to 60° C., more preferably, 25 to 50° C. In a further embodiment, the cultivation may be carried out continuously at a constant temperature. In another embodiment, the cultivation may be subject to changing temperatures.

In one embodiment, the moisture level of the mixture should be suitable for the microorganism of interest. For example, the moisture level may range from 20% to 90%, preferably, from 30 to 80%, more preferably, from 40 to 60%.

In one embodiment, the pH of the mixture should be suitable for the microorganism of interest. Buffering salts, and pH regulators, such as carbonates and phosphates, may be used to stabilize pH near an optimum value. When metal ions are present in high concentrations, use of a chelating agent in the liquid medium may be necessary.

The microbes can be grown in planktonic form or as biofilm. In the case of biofilm, the vessel may have within it a substrate upon which the microbes can be grown in a biofilm state. The system may also have, for example, the capacity to apply stimuli (such as shear stress) that encourages and/or improves the biofilm growth characteristics.

### Preparation of Microbe-Based Products

The microbe-based products of the subject invention include products comprising the microbes and/or microbial growth by-products and optionally, the growth medium and/or additional ingredients such as, for example, water, carriers, adjuvants, nutrients, viscosity modifiers, and other active agents.

The microbe-based products of the subject invention may be, for example, microbial inoculants, biopesticides, nutrient sources, remediation agents, health products, and/or biosurfactants.

One microbe-based product of the subject invention is an inoculum comprising the culture medium containing the microorganism and/or the microbial growth by-products produced by the microorganism and/or any residual nutrients. The product of cultivation method may be used directly without extraction or purification. If desired, extraction and purification can be easily achieved using standard extraction methods or techniques known to those skilled in the art.

The microorganisms in the inoculum may be in an active or inactive form. The inoculum may be used without further stabilization, preservation, and storage. Advantageously, direct usage of these inoculums preserves a high viability of the microorganisms, reduces the possibility of contamination from foreign agents and undesirable microorganisms, and maintains the activity of the by-products of microbial growth.

The inoculum can be removed from the drum and transferred via, for example, piping for immediate use.

Advantageously, in accordance with the subject invention, the inoculum may comprise broth in which the microbes were grown. The product may be, for example, at least, by weight, 1%, 5%, 10%, 25%, 50%, 75%, or 100% broth. The amount of biomass in the product, by weight, may be, for example, anywhere from 0% to 100% inclusive of all percentages there-between.

The subject invention further provides materials and methods for the production of biomass (e.g., viable cellular material), extracellular metabolites (e.g., both small and large molecules), and/or intracellular components (e.g., enzymes and other proteins). The microbes and microbial growth by-products of the subject invention can also be used for the transformation of a substrate, such as an ore, wherein the transformed substrate is the product.

The subject invention further provides microbe-based products, as well as uses for these products to achieve beneficial results in many settings including, for example, improved bioremediation and mining; waste disposal and treatment; enhancing livestock and other animal health; and promoting plant health and productivity by applying one or more of the microbe-based products.

In one embodiment, the subject invention provides a method of improving plant health and/or increasing crop yield by scaling the microbe-based product disclosed herein, for example in an on-site fermentation system, and applying the scaled product to soil, seed, or plant parts. In another embodiment, the subject invention provides a method of increasing crop or plant yield comprising multiple applications of the scaled product.

In another embodiment, the method for producing microbial growth by-products may further comprise steps of concentrating and purifying the by-product of interest.

In one embodiment, the composition is suitable for agriculture. For example, the composition can be scaled and used to treat soil, plants, and seeds. The composition may also be used as a pesticide.

In one embodiment, the subject invention further provides customizations to the materials and methods according to the local needs. For example, the method for cultivation of microorganisms may be used to grow those microorganisms located in the local soil or at a specific oil well or site of pollution. In specific embodiments, local soils may be used as the solid substrates in the cultivation method for providing a native growth environment. Advantageously, these microorganisms can be beneficial and more adaptable to local needs.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1—Mixing and Cultivation Device and Modes of Operation

A portable and distributable mixing device was constructed as shown in FIG. 1. The device has a plastic rotating drum supported by a frame having rubber wheels. Three to four baffles are attached around the inner circumference of the drum.

The rotation of the drum was powered by an electric motor connected to a power supply, allowing the drum to rotate at a speed of 15-25 rpm. The drum had a working volume of 100 liters (L) for growing Starmerella yeast for cell and metabolite production (however, size and scale can vary depending on the required application). The device is particularly well-suited for submerged culture of Starmerella clade yeast inoculums that are suitable for inoculating larger-scale on-site fermentation systems.

In order to further reduce the cost of culture production and ensure scalability of the technology, the system does not need to be sterilized using traditional methods. Instead, a method of empty vessel sanitation can be used that includes applying a highly pressurized steam stream for 10 minutes to the internal surfaces of the drum, followed by overnight treatment of the internal surfaces with 1-3% hydrogen peroxide, preferably 3% hydrogen peroxide, while rotating the drum. Additionally, in order to reduce the possibility of contamination, water used for preparing the culture can be filtered through a 0.1-micron filter.

### Nutrient Media Composition and Cultivation of Yeast Cultures

The culture medium used for producing the yeast inoculum comprised the components shown in Table 1.

TABLE 1
Components for culture medium.

| Reagent | Weight (g/L) |
|---|---|
| Yeast Extract | 5 |
| Glucose | 20 |
| Monopotassium phosphate | 2 |
| Dipostassium phosphate | 2 |
| Magnesium sulfate | 0.5 |

The culture medium components were sterilized in 1 L of 10% hydrogen peroxide overnight. The sterile composition was then mixed with filtered water in the drum of the mixer.

The cultivation temperature was generally about room temperature, from 18 to 25° Celsius. The initial pH of the medium was from about 5.5-6.0.

Under these cultivation conditions, industrially useful production of biomass, sophorolipids and other metabolites are achieved after about 1 to about 5 days of cultivation, preferably after a cultivation time of about 48 hours.

Upon completion of the cultivation, the final concentration of yeasts achieved is approximately 200 to 400 CFUs. The culture can then be used to inoculate a fermentation system, wherein the culture can be scaled for a variety of industrial purposes.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein are incorporated by reference in their entirety, including all figures and tables, to the extent they are not inconsistent with the explicit teachings of this specification.

1. A method of cultivating microorganisms, the method comprising:
providing a fermentation device comprising:
a support frame;
a rotatable drum mounted on the support frame; and
a motor connected to the drum, wherein the motor causes the drum to rotate;
adding a microorganism to the drum of the fermentation device; and
allowing the fermentation device to operate, thereby cultivating the microorganism.

2. The method according to claim 1, further comprising adding into the drum nutrients for the microorganism.

3-5. (canceled)

6. The method according to claim 1, wherein the fermentation device further comprises a plurality of baffles on an interior surface of the drum.

7-10. (canceled)

11. The method according to claim 1, wherein the fermentation device further comprises a means for adjusting an angle of the drum.

12-15. (canceled)

16. The method according to claim 1, wherein allowing the fermentation device to operate comprises allowing the device to operate continuously for a period of time of at least one day.

17. (canceled)

18. The method according to claim 1, wherein a volume of the drum is in a range of from 10 liters to 1,500 liters.

19-26. (canceled)

27. The method according to claim 1, further comprising:
before adding the microorganism to the drum, sterilizing the drum in situ.

28. (canceled)

29. The method according to claim 27, wherein the sterilizing of the drum comprises washing with hydrogen peroxide as a sterilizing agent.

30. The method according to claim 1, wherein the microorganism produces antimicrobial metabolites or byproducts, such that the fermentation device is self-sterilizing.

31-38. (canceled)

39. The method according to claim 1, wherein the microorganism is a bacterium or a fungus.

40. The method according to claim 39, wherein the microorganism is a bacterium and the bacterium is Escherichia coli, Rhizobium, Bradyrhizobium, Bacillus, Azobacter, Arhrobacter, Pseudomonas, Azospirillium, Azomonas, Derxia, Beijerinckia, Nocardia, Klebsiella, Clavibacter, cyanobacteria, Pantoea, Sphingomonas, Streptomyces, Streptoverticillium, Ralslonia, Rhodospirillum, Xanthomonas, Erwinia, or Clostridium.

41. The method according to claim 39, wherein the microorganism is a fungus and the fungus is Starmerella, Mycorrhiza, Mortierella, Phycomyces, Blakeslea, Thraustochytrium, Penicillium, Phythium, Entomophthora, Aureobasidium pullulans, Fusarium venenalum, Aspergillus, Trichoderma, Rhizopus spp, endophytic fungus, Saccharomyces, Debaromyces, Issalchenkia, Kluyveromyces, or Pichia spp.

42. (canceled)

43. The method according to claim 39, wherein the microorganism is Mycorrhizal fungus or Starmerella fungus.

44-53. (canceled)

54. A composition comprising the microorganism cultivated by the method according to claim 1 and/or at least one microbial growth by-product of said microorganism.

55. A fermentation device for cultivating microorganisms, the device comprising:
a support frame;
a rotatable drum mounted on the support frame;
a plurality of baffles on an interior surface of the drum;
at least one wheel attached to a lower portion of the support frame; and
a motor connected to the drum, wherein the motor causes the drum to rotate.

56. The device according to claim 55, wherein the motor is an electric motor or a gas-powered motor.

57. The device according to claim 55, further comprising a battery to which the motor is connected.

58. The device according to claim 55, wherein the motor is configured to be connected to an external power source during operation.

59. The device according to claim 55, comprising a plurality of wheels at the bottom portion of the frame.

60. The device according to claim 55, further comprising a means for adjusting an angle of the drum.

61-64. (canceled)

65. The device according to claim 55, wherein the drum has a shape of a cylinder or a modified cylinder.

66. The device according to claim 55, wherein a volume of the drum is in a range of from 10 liters to 1,500 liters.

67-68. (canceled)

69. The device according to claim 55, further comprising a temperature sensor for measuring temperature within the drum and a pH sensor for measuring pH within the drum.

70. (canceled)

71. The device according to claim 55, wherein the fermentation device further comprises: an oxygen sensor for measuring dissolved oxygen within the drum; an agitation sensor for measuring agitation within the drum; a foaming sensor for measuring foaming within the drum; a microbial culture sensor for measuring purity of microbial cultures within the drum; a metabolite sensor for measuring production of desired metabolites within the drum; or a combination thereof.

72. (canceled)

73. The device according to claim 55, further comprising a sterilizing unit for sterilizing the drum in situ.

74-86. (canceled)
